(19) United States
(12) Patent Application Publication
(10) Pub. No.: US 2012/0009277 A1
(43) Pub. Date: Jan. 12, 2012

(54) METHOD FOR SELECTION OF NOVEL ANTI-CANCER HERBS USING CHEMINFORMATIC TOOLS

(76) Inventors: Soroush Sardari, Tehran (IR); Ghazaleh Ghavami, Tehran (IR)

(21) Appl. No.: 12/831,283

(22) Filed: Jul. 7, 2010

(51) Int. Cl. G06F 17/30; A61P 35/00; A61K 33/24; A61K 36/605; A61K 36/30

(52) U.S. Cl. 424/649; 424/769; 424/725; 707/722; 707/E17.09; 707/E17.014; 707/769; 707/740

(57) ABSTRACT

The various embodiments herein provide a new method of selecting novel anti-cancer herbs using cheminformatics tools. The method involves collecting the herbs having synergistic activity with anti-cancer compounds and categorizing the herbs as synergistic plants (SP). The bioactive compounds are selected from the SP and categorized into synergistic compounds collection (SCC). A similarity search is performed using the selected bioactive compounds through available databases to select similar compounds that are categorized as similar synergistic compounds (SSC). A herbal source for the SSC is searched and categorized as similar herbal synergistic compounds (SHSC). The novelty of the SHSC in cancer treatment is confirmed. The novel herbs are categorized as novel candidate herbs (NCH). The synergistic activity of the resulted herbs (NCH) is confirmed by performing in vitro bioassay.

### BACKGROUND

1. Technical Field

The embodiments herein generally relate to a field of cheminformatics and more particularly to a method for selecting anticancer herbs using the cheminformatic tools.

2. Description of the Related Art

Cheminformatics is a new born science which joins computational science to chemistry and biology and has been applied in modern drug discovery to enhance the success rate from a systematic knowledge use. Cheminformatics methodology plays magic roles in several scientific researches such as basic chemistry and biology research, drug discovery, drug design, industrial pharmacological researches, medicinal chemistry researches, diagnosis, etc. The various applications of cheminformatics in drug discovery researches can be summarized in eight categories: data mining, chemical structure representation, similarity and diversity search, analysis of data from analytical chemistry, property predictive model presentation, computer-assisted structure elucidation (CASE), computer-assisted synthesis design (CASD), and molecular modeling.

Similarity and diversity search is a standard cheminformatics tool in drug discovery area and database designing. Molecular similarity is using various parameters widely applied in database searching for acquiring compounds and generating libraries. Molecular similarity analysis presents the way of molecules to cover a determined structural space and underlies many approaches for compound selection and design of combinatorial libraries. The choice of an optimal metric space that bitterly represents the structural diversity of a compound population is determinant in the efficiency of the model. A correct diversity/similarity space should allow us to place molecules in good position relative to others in a well parameterized way. The computation time of the similarities and the diversities between compounds is equally important, due to the growing popularity of big databases.

Similarity search is one of the most efficient methods in cheminformatics to detect specific biomolecular target, which is expected to have an activity for a given disease such as cancer. Molecular similarity analysis delves the way for molecules to cover a determined structural space and underlies many approaches for compound selection and design of combinatorial libraries. A proper diversity/similarity space put molecules in good position relative to others in a well parameterized way. Three main parameters in similarity search are the descriptors, the coefficients and the weighting scheme. Each molecule in similarity search process is identified with an ID code; one of the most common molecular structure codes is SMILES (Simplified Molecular Input Line Entry Specification). The General approach involves the use of a set of algorithms to compare a query sequence to all the sequences in a specified database. Comparisons are made in a pair wise fashion. Each comparison is given a score reflecting the degree of similarity between the query and the sequence being compared. The higher the score, the greater the degree of similarity.

Treatment of cancer involves injuring or killing of normal body cells as undesirable side effects. On the other side, a major impediment in anticancer chemotherapy process is Multidrug resistance (MDR) phenomenon. This phenomenon relates to the property of drug resistant tumors to exhibit simultaneous resistance to a number of structurally and functionally unrelated chemotherapeutic agents. The three biological mechanisms involved in MDR phenomenon are: physiological properties of tumor cells, non-classical MDR mechanisms, and transport-based classical MDR mechanisms. Increase of environmental interstitial fluid pressure, high vascular permeability and acidic environment are some of the physiological and hematological properties of tumor cells which lead to the reduction of effect of anticancer agent on tumor cells. The non-classical MDR mechanisms involve change in the balance of proteins that control apoptosis, such as p53, Bcl-2. It also involves altered activity of specific enzyme systems such as S-transferase, GST, cyclooxygenase-2 and topoisomerase. The transport-based classical MDR mechanisms involves the over-expression or activation of transmembrane proteins that efflux different chemical substances from the tumor cells. These transporter proteins are typically named as ATP-binding cassette (ABC) family which can reduce intracellular accumulation of drugs. Most typical efflux pumps over-expressed in the membrane of tumor cells are multidrug resistant associated protein (MRP) and P-Glycoprotein (P-GP). Today, wide ranges of scientific efforts are focused on blocking and reversal of these ABC transporters involved in transport-based classical MDR mechanisms to overcome MDR phenomenon.

One approach to overcome the MDR phenomenon is combinational drug therapy. Combinational drug therapy has a long history and its roots can be found in traditional Chinese medicines. Today parallel to new advances in cancer chemotherapy, cancer combinational drug therapy has been developed extremely. Wide ranges of scientific efforts are focused on reversing efflux of anticancer agents from tumor cells; increasing the efficacy; reducing the dosage but increasing or maintaining the efficacy of cytotoxic agent; reducing or overcoming drug resistance phenomena; and achieving selective synergism against target or toxicity antagonism. Synergistic, additive and antagonistic effects are the three forms of combinational drug therapy effects on tumor cells. Consequently, it seems that combination therapy may be able to overcome cancer chemotherapy side effects by decreasing drug effective dose and increasing protection of normal cells against antitumor drugs. Wide ranges of scientific efforts are focused on combination therapy to be used in cancer treatment to induce efficacy, reduce or overcome drug resistance phenomena and achieve selective synergism against target or toxicity antagonism.

Use of cytotoxic agents in cancer combinational drug therapy have been categorized in five different sets: firstly, cytotoxic agents in combination with cytotoxic drugs, for example, edatrexate and cisplatin, paclitaxel (Taxol) and doxorubicin (Adriamycin), temozolomide and didox, temozolomide and other cytotoxic agents, discodermolide and paclitaxel, didox and carmustine, oral proteosome inhibitor and bortezomib, oxaliplatin and CPT-11, gemcitabine and various antitumor agents, irinotecan and 5-FU and oxaliplatin ternary combination, XR 5944 and carboplatin or doxorubicin. Secondly, cytotoxic agents in combination with cyto-differentiating agents. Thirdly, cytotoxic agents in combination with MDR reversing agents, for example, carboplatin resistance, MDR protein resistance reversal by ardeemins, MDR reversal by ningalins, nordihydroguaiaretic acids and doxorubicin or paclitaxel. Forthly, cytotoxic agents in combination with modulators, for example, retinoic acid, GCSF and LiCl or all-trans-retinoic acid, IFNα and lovastatin/bcr-abl and cells. Fifthly, Cytotoxic agents in combination with virus and enzymes for example, trimetrexate and carboxypeptidase G2.

Use of chemosensitizers in combination with anticancer drugs, is another approach to overcome MDR phenomenon, whereby leading to sensitize MDR tumor cells, reversing and inhibiting ABC transporters involved in multidrug resistance phenomenon. Verapamil, cyclosporin A, rapamycin, and PSC-833, VX-710, LY335979, XR9051, XR9576 and flavonoid kaempferide are some of chemosensitizers that are applied clinically. Doxorubicin and cisplatin are used widely in cancer chemotherapy but unfortunately as exogenous substrate for ABC transporters (doxorubicin; MDR1, MRP2, MRP3, MRP5 and MRP6 transporters, cisplatin; MRP2 and MRP3 transporters), which are exposed by MDR pumps from tumor cells and cannot play efficient roles in chemotherapy.

As the drug discovery process has become more expensive and the computation time in bringing up these discoveries is large. Hence there is a need for a novel methodology which uses less time, has better cost effectiveness and plays magic roles in several biomedical and clinical scientific researches.

The above mentioned shortcomings, disadvantages and problems are addressed herein and which will be understood by reading and studying the following specification.

### Objectives of the Invention

The primary object of the embodiments herein is to provide a novel logical algorithm by focusing on similarity searching tools to acquire novel candidate herbs with promising profiles.

Another object of the embodiments herein is to provide herbs containing similar herbal synergistic compounds with novelty profile in cancer therapy studies.

Yet another object of the embodiments herein is to provide the utilization of in silico methods in biomedical research area to enhance the chances of success in discovery processes.

Yet another object of the embodiments herein is to provide the use of in silico methods for identifying and generating the best lead drug candidates.

Yet another object of the embodiments herein is to provide the use of in silico methods which uses less time and has better cost effectiveness.

Yet another object of the embodiments herein is to provide a novel approach by combining cheminformatics, intensive literature handling together with correlation of biologic data to search for the desired biologic activity in the domain of natural products that are not explored before.

Yet another object of the embodiments herein is to provide a novel methodology which can be successfully used in the area of cytotoxic agents for the discovery lead drugs.

These and other objects and advantages of the embodiments herein will become readily apparent from the following detailed description taken in conjunction with the accompanying drawings.

### SUMMARY

The various embodiments herein provide a new method of selecting novel herbs/drugs using cheminformatics tools. According to an embodiment a method of selecting novel herbs comprising the steps of collecting a pluralities of herbs with a property to treat a desired disease from a herbal medicinal database. The collected pluralities of herbs are classified into synergistic plants. Pluralities of bioactive compounds are selected from the synergistic plants. The selected bioactive compounds categorized into synergistic compounds. Simplified Molecular Input Line Entry Specification (SMILES) are acquired based on the chemical structures of the synergistic compounds. A similarity search is conducted using the acquired SMILES as a data query to find and classify compounds that are similar to categorize the synergistic compounds into similar synergistic compounds. The similar synergistic compounds that are derived from a herbal source are classified into similar herbal synergistic compounds the novelty of the herbs containing similar herbal synergistic compounds in treating a desired disease is checked. A synergistic property of the herbs containing similar herbal synergistic compounds is confirmed through a bioassay process to determine the synergistic property of the herbs containing similar herbal synergistic compounds in treating the desired disease. The herbs containing similar herbal synergistic compounds are grouped into candidate herbs based on the determined synergistic property with respect to the desired disease.

The similar synergistic compounds are searched and acquired using an algorithm. The algorithm is derived based on a cheminformatics tool. The herbal medicinal database includes previous findings. The desired disease is cancer.

The pluralities of herbs with a property to treat a desired disease are collected by searching the pluralities of herbs with the property to treat the desired disease from the previous findings. The previous findings include available books and articles. The novelty of the herbs containing similar herbal synergistic compounds in treating the desired disease is checked by performing a literature survey. The synergistic property of the herbs containing similar herbal synergistic compounds is confirmed by performing in vitro assay to confirm a promising profile of the herbs containing similar herbal synergistic compounds in treating the desired disease.

According to an embodiment, a method of selecting novel herbs comprising the steps: selecting compounds with natural source from previous findings wherein the previous findings consists of available herbal databases, books and articles; conducting similarity search using the selected compounds; conducting literature search for the compounds obtained from the similarity search; and selecting compounds having promising profiles resulted from the literature search.

According to one embodiment of the present invention, a new method of selecting novel herbs/drugs using cheminformatics tools, wherein the method comprises: collecting herb reported to have desired property of treating a disease by searching available herbal medicine databases, books and articles; selecting a bioactive compound from the collected herb having the desired property; performing similarity search using the bioactive compound as input query; selecting similar compound from an output result generated; searching herbal source for the selected similar compound; and performing in vitro bioassay in order to confirm the desired property of the obtained herbs/drugs, thus providing an in silico method for drug discovery process in order to identify and generate the best lead drug candidates.

According to another embodiment of the present invention, a new method of selecting novel anti-cancer herbs using cheminformatic tools, wherein the method comprising the steps: collecting herbs reported to have synergistic activity with anti-cancer compounds by searching in herbal medicine databases and previous findings and categorizing the above herbs as synergistic plants (SP); selecting bioactive compounds from the synergistic plants (SP) and categorizing as synergistic compounds collection (SCC); performing similarity search using the selected bioactive compounds categorized as SCC through available databases;

obtaining a result and selecting compounds from the result having similarity with the selected bioactive compound and categorizing as similar synergistic compounds (SSC); searching herbal source for the similar synergistic compounds (SSC) and categorizing compounds having the herbal source as similar herbal synergistic compounds (SHSC); looking for novelty of the herbs obtained as SHSC in field of cancer treatment; categorizing the herbs which are novel as novel candidate herbs (NCH); and performing in vitro bioassay in order to confirm the synergistic activity of the resulted herbs (NCH) with anti-cancer agents, and thus providing an in silico method for discovering a drug in field of cancer.

According to another embodiment of the present invention, a new method of finding novel herbs/drugs working in synergism with each other using cheminformatic tools, wherein the method comprising the steps: collecting herbs reported to have synergistic activity with each other by searching in herbal medicine databases and previous findings and categorizing the above herbs as synergistic plants(SP); selecting bioactive compounds from the synergistic plants (SP) and categorizing as synergistic compounds collection (SCC); performing similarity search using the selected bioactive compounds categorized as SCC through available databases; obtaining a result and selecting compounds from the result having similarity with the selected bioactive compound and categorizing as similar synergistic compounds (SSC); searching herbal source for the similar synergistic compounds (SSC) and categorizing compounds having the herbal source as similar herbal synergistic compounds (SHSC); looking for novelty of the herbs obtained as SHSC; categorizing the herbs which are novel as novel candidate herbs (NCH); and performing in vitro bioassay in order to confirm the synergistic activity of the resulted herbs(NCH), and thus providing an in silico method for discovering a drug in field of cancer.

According to another embodiment of the present invention, a composition for treating cancer, wherein the composition comprises: herbal extracts of Morus alba, Musa sapientum, Arnebia decumbens, Arnebia echioides, Arnebia linearifolia DC in combination with doxorubicin and cisplatin.

According to another embodiment of the present invention, a composition for treating a patient suffering from cancer comprising herbal extracts of Morus alba, Musa sapientum, Arnebia decumbens, Arnebia echioides, Arnebia linearifolia DC working in synergism/combination with anticancer agents, wherein the anticancer agents are cisplatin and doxorubicin.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flow chart explaining the method of selecting herbs for combination drug therapy using an algorithm, according to one embodiment herein.

FIG. 2 illustrates a flow chart explaining the various steps for selecting novel anti-cancer herbs using cheminformatic tools according to one embodiment herein.

FIG. 3 shows the chemical structures for kaempferol, epicatechin and juglone used as input material for similarity search according to one embodiment herein.

FIG. 4 shows the chemical structures for morin, leucocyanidin, arnebin 7 and arnebin generated as output from similarity searching according to one embodiment herein.

FIG. 5 shows a graphical representation of the synergistic property of candidate herbal extracts with cisplatin using A2780-cp cell line according to one embodiment herein.

FIG. 6 shows a graphical representation of the synergistic property of candidate herbal extracts with doxorubicin using ACHN cell line according to one embodiment herein.

FIG. 7 shows a graphical representation of the antagonistic property of candidate herbal extracts with doxorubicin using HF2 cell line according to one embodiment herein.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a new method of selecting novel herbs using cheminformatics tools, wherein the method comprising the steps: selecting compounds with natural source from previous findings wherein the previous findings consists of available herbal databases, books and articles; conducting similarity search using the selected compounds; conducting literature search for the compounds obtained from the similarity search; and selecting compounds having promising profiles resulted from the literature search.

According to one embodiment of the present invention, a new method of selecting novel herbs/drugs using cheminformatics tools, wherein the method comprises: collecting herb reported to have desired property of treating a disease by searching available herbal medicine databases, books and articles; selecting a bioactive compound from the collected herb having the desired property; performing similarity search using the bioactive compound as input query; selecting similar compound from an output result generated; searching herbal source for the selected similar compound; and performing in vitro bioassay in order to confirm the desired property of the obtained herbs/drugs, thus providing an in silico method for drug discovery process in order to identify and generate the best lead drug candidates.

According to another embodiment, a new method of selecting novel anti-cancer herbs using cheminformatic tools, wherein the method comprising the steps: collecting herbs reported to have synergistic activity with anti-cancer compounds by searching in herbal medicine databases and previous findings and categorizing the above herbs as synergistic plants (SP); selecting bioactive compounds from the synergistic plants (SP) and categorizing as synergistic compounds collection (SCC); performing similarity search using the selected bioactive compounds categorized as SCC through available databases; obtaining a result and selecting compounds from the result having similarity with the selected bioactive compound and categorizing as similar synergistic compounds (SSC); searching herbal source for the similar synergistic compounds (SSC) and categorizing compounds having the herbal source as similar herbal synergistic compounds (SHSC); looking for novelty of the herbs obtained as SHSC in field of cancer treatment; categorizing the herbs which are novel as novel candidate herbs (NCH); and performing in vitro bioassay in order to confirm the synergistic activity of the resulted herbs(NCH) with anti-cancer agents, and thus providing an in silico method for discovering a drug in field of cancer. The resulted novel candidate herbs (NCH) are Morus alba, Musa sapientum, Arnebia decumbens, Arnebia echioides, Arnebia linearifolia DC. The anti-cancer agents used are doxorubicin and cisplatin. The bioactive compounds selected from the synergistic plants (SP) and categorized as synergistic compounds collection (SCC) are kaempferol, epicatechin and juglone. The compounds selected from the obtained result and categorized as similar synergistic compounds (SSC) are morin, leucocyanidin, arnebin 7 and arnebin.

According to another embodiment, a new method of finding novel herbs/drugs working in synergism with each other using cheminformatic tools, wherein the method comprising the steps: collecting herbs reported to have synergistic activity with each other by searching in herbal medicine databases and previous findings and categorizing the above herbs as synergistic plants (SP); selecting bioactive compounds from the synergistic plants (SP) and categorizing as synergistic compounds collection (SCC); performing similarity search using the selected bioactive compounds categorized as SCC through available databases;

obtaining a result and selecting compounds from the result having similarity with the selected bioactive compound and categorizing as similar synergistic compounds (SSC); searching herbal source for the similar synergistic compounds (SSC) and categorizing compounds having the herbal source as similar herbal synergistic compounds (SHSC); looking for novelty of the herbs obtained as SHSC; categorizing the herbs which are novel as novel candidate herbs (NCH); and performing in vitro bioassay in order to confirm the synergistic activity of the resulted herbs(NCH), and thus providing an in silico method for discovering a drug in field of cancer.

According to another embodiment, a composition for treating cancer, wherein the composition comprises: herbal extracts of Morus alba, Musa sapientum, Arnebia decumbens, Arnebia echioides, Arnebia linearifolia DC in combination with doxorubicin and cisplatin.

According to another embodiment, a composition for treating a patient suffering from cancer comprising herbal extracts of Morus alba, Musa sapientum, Arnebia decumbens, Arnebia echioides, Arnebia linearifolia DC working in synergism/combination with anticancer agents, wherein the anticancer agents are cisplatin and doxorubicin.

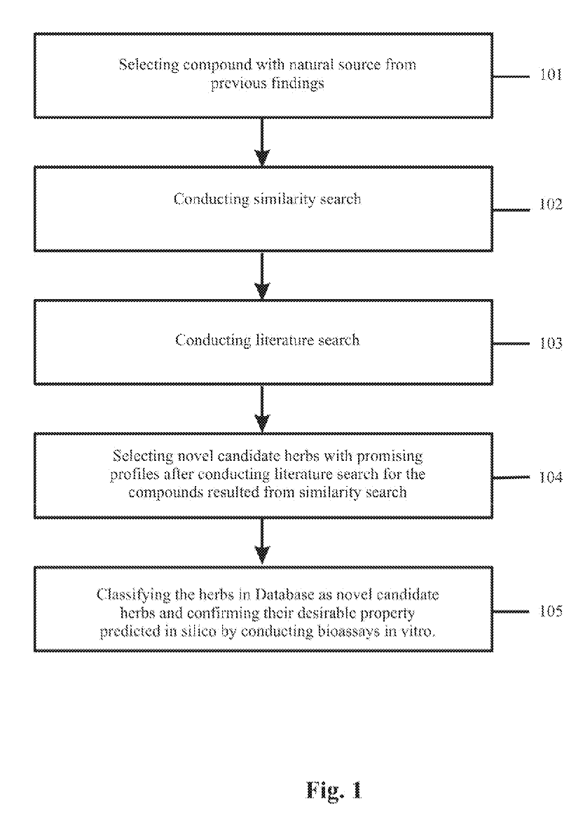

FIG. 1 illustrates a flow chart explaining the plant selection process wherein showing a logical algorithm focusing on molecular similarity searching tools designed to discover and present novel candidate herbs with promising profiles, according to one embodiment herein. With respect to FIG. 1, compounds are selected with a natural source from previous findings (101). The compounds are searched in available herbal medicine databases, books and articles. The findings are categorized in Database 1. The bioactive compounds with respected or desired property derived from Database 1 are gathered in Database 2. Chemical structures of the compounds are drawn by Pubchem server to obtain SMILES (Simplified Molecular Input Line Entry Specification) as a data query for molecules to do similarity search. Similarity search is conducted (102). Mainly, two cheminformatics servers like http://pubchem.ncbi.nlm.nih.gov/search/search.cgi; http://cactus.nci.nih.gov/ncidb2 are used to perform similarity search on the basis of “Tanimoto index X % scores” (i.e. score>=X %). The resulted similar compound is classified in Database 3. The compounds are searched for their herbal source and gathered in database 4. Literature search is conducted for the herbs (103). Literature search is conducted for novelty of the herbal sources of the resulted compounds in the field of respected property. Literature search is conducted in sources like www.google.com www.scirus.com, www.sciencedirect.com and www.ncbi.nlm.nih.gov. Novel candidate herbs with promising profiles are selected after conducting literature search (104). The results are classified in Database 5 as novel candidate herbs and their desirable property is confirmed by conducting in vitro bioassays predicted by in silico method (105).

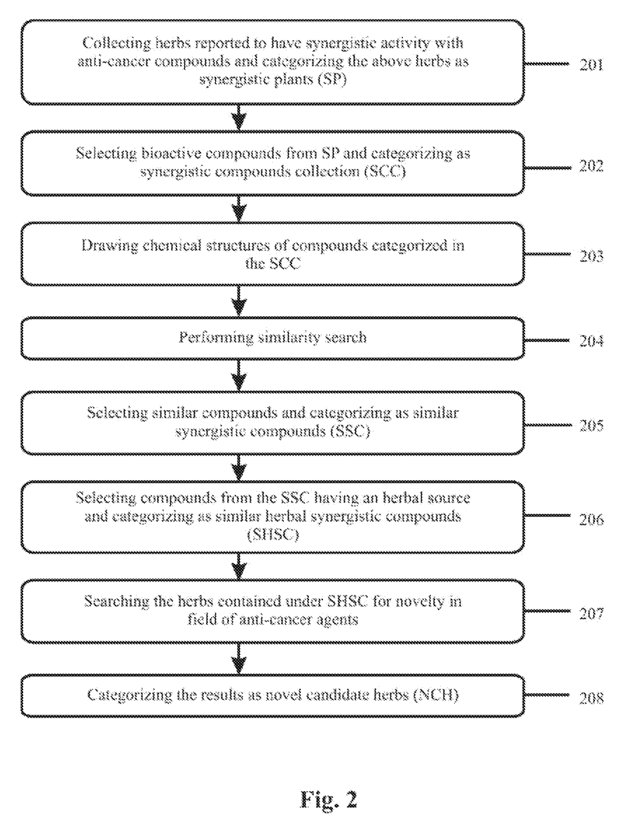

FIG. 2 illustrates a flow chart explaining the various steps for selecting novel anti-cancer herbs using cheminformatic tools, according to one embodiment herein. With respect to FIG. 2, the herbs reported to have synergistic activity with anti-cancer compounds are collected. The synergistic activity searched in herbal medicine databases and previous findings i.e. books and articles. The herbs are categorized as synergistic plants (SP) (201). Bioactive compounds are selected from the synergistic plants (SP) and categorized as synergistic compounds collection (SCC) (202). The chemical structures of compounds categorized in the SCC are drawn (203). The chemical structures are drawn from Pubchem server. SMILES (Simplified Molecular Input Line Entry Specification) codes are obtained in order to use as input material for similarity search. Similarity search is performed (204). Mainly two servers are used for similarity search. They are http://pubchem.ncbi.nlm.nih.gov/search/search.cgi and http://cactus.nci.nih.gov/ncidb2. Similarity search is performed on the basis of Tanimoto index X % scores. Compounds having the score equal or more than 95% are selected and categorized as similar synergistic compounds (SSC) (205). Compounds having a herbal source are selected from the similar synergistic compounds (SSC) and categorized as similar herbal synergistic compounds (SHSC) (206). The herbs contained under SHSC are searched for novelty in field of anti-cancer agents (207). The results are categorized as novel candidate herbs (NCH) (208). In vitro bioassays are performed to confirm synergistic activity of the resulted herbs with anti-cancer agents. This provides an in silico method for drug discovery process in order to identify and generate the best lead drug candidates.

The novel candidate herbs (NCH) obtained are Morus alba, Musa sapientum, Arnebia decumbens, Arnebia echioides, Arnebia linearifolia DC. Anti-cancer agents are doxorubicin and cisplatin.

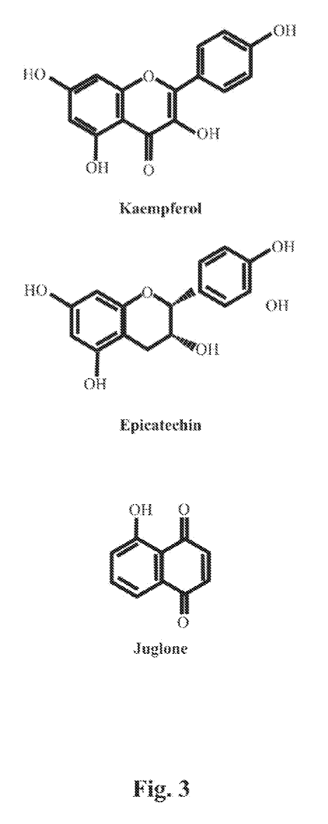

FIG. 3 shows the chemical structures for kaempferol, epicatechin and juglone used as input material for similarity search, according to one embodiment herein.

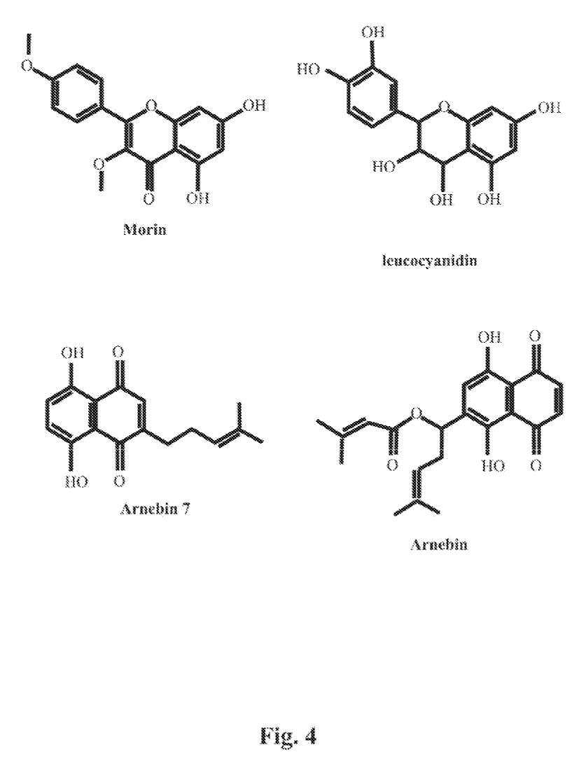

FIG. 4 shows the chemical structures for morin, leucocyanidin, arnebin 7 and arnebin generated as output from similarity searching according to one embodiment herein. Kaempferol, epicatechin and juglone are the input material for similarity search, which are used to present morin, leucocyanidin, arnebin 7 and arnebin as output result. A computational algorithm is designed herein. Two online servers http://pubchem.ncbi.nlm.nih.gov/search/search.cgi and http://cactus.nci.nih.gov/ncidb2 used in this study for conducting similarity search have predicted antitumor enhancer, cyclooxygenase-2 inhibitory and MDR pumps blocking effects for mentioned five candidate herbs.

Morin and leucocyanidin are two output phytocompounds that are structurally similar to kaempferol as an ABC transporter inhibitor and epicatechin as a cyclooxygenase-2 and ABC transporter inhibitor with “Tanimoto index 98%”, also arnebin and arnebin 7 are another outputs that are similar to juglone as an antineoplastic enhancer (synergistic effect with antitumor drug) with “score >=95%” structurally. On the basis of proved theory presented in which compounds with similar chemical structure show similar chemical properties, it is predicted that the output compounds with similarity rate of 95% to our input phytocompounds, have showed similar pointed properties and reduced MDR phenomena. This logical prediction performed by cheminformatics tools in silico is tested in vitro and the results further confirm the in silico method and predictions. In addition, the introduced results are useful in finding applications for the effect of these herbs in reversing MDR phenomenon.

Cancerous cell lines ACHN and A2780/cp is selected to determine combination effects of mentioned herbal extracts with doxorubicin and cisplatin by MTT colorimetric assay. On the basis of previous results, a major cause of cancerous cell resistance to doxorubicin is over expression of MDR1, MRP2, MRP3, MRP5 and MRP6 transporters in MDR cell lines, NCI database www.dtp.nci.nih.gov findings have reported MDR1 and LRP pumps have been over expressed in 31.0 and 1.50 levels in ACHN cell line. Therefore, ACHN cell line has been selected in determining the combination effects of five herbal extracts with doxorubicin for reversing MDR phenomenon. MRP2 and MRP3 transporters are two sub types of proton-transporting ATPases. Efflux of these MDR pumps cause resistance of cancerous cell lines to cisplatin. Analyses of A2780/cp (standard cancerous ovarian cell lines resistant to cisplatin) protein pattern have shown that proton-transporting ATPases and ATP synthase complexes that play key role in MDR phenomenon are enhanced and over-expressed significantly. Human fetal cell line (HF2) as a normal cell line (which does not express MDR efflux pumps is selected as a cell negative control in comparison with ACHN to determine combinational treatment effects of five herbal extracts (in several concentrations) with doxorubicin.

The IC 50 value is calculated for doxorubicin and cisplatin, alone and in combination with each five herbal extracts in concentrations of 100, 50, 25 and 12.5 μg/ml. The Chemosensitizing index (CSI) is also calculated by dividing IC50 value for drug alone with IC50 value for drug plus herbal extract in order to indicate difference between cytotoxicity induction of anticancer drugs alone and in combination with herbal extracts. To analyze the interaction between herbal extracts and the anticancer drugs, Zheng-Jun Jin and CI methods are applied. Hemolytic test is performed to confirm the bio-compatibility of these herbs with anti-cancer agents. Hemolytic and cytotoxic experiment results are expressed as mean±SD. Mean difference among groups is calculated by One-way and Repeated measures ANOVA. p<0.05 is considered significant statistically. The results show that, all five herbal extracts synergistically increased cytotoxic effects of cisplatin and doxorubicin on A2780/cp and ACHN in dose dependent manner. Since MDR efflux pumps are expressed on the cell membranes of HF2 cells and this cell line is not resistant to doxorubicin, all five herbal extracts antagonistically decrease cytotoxic effect of doxorubicin on HF2 as normal cell. Both resulted “Q” and “CI” values calculated by two mentioned methods show synergistic effect of combination treatment in A2780/cp and ACHN, but effect of combination treatment on HF2 followed an antagonistic profile.

Means of cytotoxic percents induced by single anticancer drug treatments and combinational treatments (several concentrations of five herbal extracts in combination with anticancer drugs) are calculated and results are compared by One-way Analysis of Variance (ANOVA) method. Findings showed the cytotoxic percents induced by single anticancer drugs; doxorubicin and cisplatin are extremely lower than the cytotoxic percents induced by combinational treatments on ACHN and A2780/cp, but the cytotoxic percents induced by single doxorubicin are extremely higher than the cytotoxic percents induced by combinational treatments on HF2 (p value<0.0001).

### Experimental Data

Experiments with one normal fibroblast cell line HF2 and two cancerous cell lines ACHN and A2780/cp exposed to combinations of herbal extracts and cytotoxic drugs are performed. Later, the cell lines are treated with herbal extracts and anticancer drugs at the same time.

Selection of candidate herbs are carried out by logical algorithm based on cheminformatics tools. The herbs reported to have synergistic activities with anticancer compound are collected by searching in herbal medicine databases and previous findings, and then findings are categorized as SP (synergistic plants). Bioactive compounds from pointed herbs are selected and categorized in SCC (synergistic compounds collection). Chemical structures of herbal synergistic compounds are drawn by Pubchem server to obtain SMILES (Simplified Molecular

Input Line Entry Specification) as a data query for molecules to carry out the similarity search. The cheminformatics-servers used to do a similarity search are http://pubchem.ncbi.nlm.nih.gov/search/search.cgi; http://cactus.nci.nih.gov/ncidb2 Compounds with “Tanimoto index 98%” and compounds with score >=95% are found similar to the first set of synergistic compounds SCC and are classified in SSC (similar synergistic compounds). SSC derived from herbal source is gathered in SHSC (similar herbal synergistic compounds). Herbs containing SHSC are searched in sources like www.google.com, www.scirus.com, www.sciencedirect.com and www.ncbi.nlm.nih.gov for the novelty item in the field of anticancer study. On the other hand, the herbs are checked by www.gbif.net, “A dictionary of Iran's vegetation plants” and “Flora of Iran” to study their existence and accessibility profile in Iran. The results are classified in NCH (novel candidate herbs) to bioassay and determine their synergistic property with anticancer drugs in vitro.

Vegetation regions of candidate herbs are guessed by searching “A dictionary of Iran's vegetation plants” and “Flora of Iran” and then candidate herbs are collected during three collection trips from North, North-West, South-West, and East of Iran. Voucher herbarium samples of collected herbs are prepared and available.

On the base of our designed algorithm by screening of the pooled data resulted from similarity search, the compounds with promising profile of availability and herbal source are selected and collected as similar herbal synergistic compounds. Herbs containing similar herbal synergistic compounds with novelty profile in cancer therapy studies are selected as novel herbal candidate to bioassay and determine their synergistic property with anticancer drugs. In brief, herbs containing chemical compounds resulted from similarity search are filtered by novelty and availability and candidate plants are collected and prepared for the bioassays as shown in Table 1.

TABLE 1
SHOWING THE NOVEL LOGICAL SELECTION OF CANDIDATE HERBS

| | Compound selected | | | Novel candidate herbs con- |
|---|---|---|---|---|
| S. | from previous | | Compound resulted from | taining compounds resulted |
| No | findings | Similarity search server | similarity search | from similarity search |
| 1. | Kaempferol | http://pubchem.ncbi.nlm. | Morin | Morus alba |
| | | nih.gov/search/search.cgi | | |
| 2. | Epicatechin | http://pubchem.ncbi.nlm. | Leucocyanidin | Musa sapientum |
| | | nih.gov/search/search.cgi | | |
| 3. | Juglone | http://cactus.nci.nih.gov/ | Arnebin 7 | Arnebia |
| | | ncidb2 | | decumbens |
| 4. | Juglone | http://cactus.nci.nih.gov/ | Arnebin | Arnebia echioides |
| | | ncidb2 | | |
| 5. | Juglone | http://cactus.nci.nih.gov/ | Arnebin | Arnebia |
| | | ncidb2 | | linearifoliaDC. |

Plant materials are obtained and dried at room temperature for 4-7 days. Dried herbal powder is extracted by ethanol (Merck, Germany) (80%) according to percolation method. The samples are steeped in solvent in the percolator for 24 hours before each extraction and the process is repeated three times. The extracts are then collected and pooled. Table 2 shows the collection and preparation of the five candidate herbal extracts.

TABLE 2
SHOWING THE COLLECTION AND PREPARATION
OF THE FIVE CANDIDATE HERBAL EXTRACTS

| | Voucher | | | | |
|---|---|---|---|---|---|
| S. | specimen | | | Collection | |
| No | number | Scientific name | Family | region | Used parts |
| 1. | 74-38 | Morus alba | Moraceae | South-West | Green leaves, fruit |
| | | | | of Iran | and woody branches |
| 2. | 77-38 | Musa | Musaceae | North | Unripe plantain |
| | | sapientum | | of Iran | banana pulp |
| 3. | 76-2 | Arnebia | Boragina- | East | Green leaves, stem |
| | | decumbens | ceae | of Iran | and flower |
| 4. | 77-29 | Arnebia | Boragina- | North-West | Green leaves, stem |
| | | echioides | ceae | of Iran | and flower |
| 5. | 85-52 | Arnebia | Boragina- | South-West | Green leaves, stem |
| | | linearifolia DC. | ceae | of Iran | and flower |

After evaporating, each extract (10 mg) is dissolved in 1 ml ethanol (50%) as stock. Doxorubicin (50 mg/25 μl) and cisplatin (50 mg/100 μl) are obtained from EBEWE Pharma Ges.m.b. Nfg. KG. A-4866 Unterach, AUSTRIA and are diluted with RPM (10% FBS).

Anticancer drug resistant cancerous cell lines; A2780/cp (human ovarian carcinoma, resistant to cisplatin) NCBI C454, ACHN (human renal adenocarcinoma) NCBI C206, and normal cell line HF2 (human fetal fibroblast) NCBI C336 herein used as a cell negative control are obtained from National Cell Bank of Iran, Pasture Institute of Iran (Tehran). These cells are maintained in RPMI 1640 medium supplemented with 10% FCS in a humidified incubator (37° C. and 5% CO₂).

Cells are cultured in RPMI-1640 medium supplemented with 10% heat inactivated FCS, 2 mM glutamine, penicillin (100 IU/μl) and streptomycin (100 μg/ml) at 37° C. in an incubator containing 5% CO₂.

Harvested cells with trypsin (0.25%) are counted by Neubauer slide and then are seeded into 96-well plates (104 cell/well). At the first step, for determining the cytotoxic property of samples, the cells are incubated with 100 μl of different concentration of ethanol herbal extracts (100, 50, 25 and 12.5 μg/ml) diluted by RPMI (10% FBS) containing doxorubicin (8, 4, 2, 1 and 0.5 μM) or cisplatin (41, 20.5, 10.250, 5.125 μM) and ethanol (50%) from 1% to 0.12% as solvent control for 24 hr. Twelve cultured wells without any treatments are used as negative control. At the same time, for studying the synergistic property, 50 μl of each ethanol herbal extract plus 50 μl of either anticancer drug in maintained concentration are added to wells [serial dilution is performed by RPMI, 10% FBS]. Each single dose and combination is in triplicate in each assay. For MTT (to assess the viability, cell counting and the proliferation of cells) assay, the content of each well is taken out and 100 μl of MTT tetrazolium dye (5 mg/ml in PBS) is added to each well and incubated at 37° C. for 3 hr. The insoluble formazan produced is dissolved in solution containing 100 μl DMSO (Dimethyl sulfoxide) and Optical Density (absorbance) is read against blank reagent with Multiwell scanning spectrophotometer (ELISA reader, Organon Tekninka, the Netherlands) at a wavelength of 545 nm. The percentage of cytotoxicity is calculated according to following equations:

$$\% Viability = ( \frac{mean absorbance of treated cells}{mean absorbance of negative control} ) \times 100$$

$$\% Cytotoxicity = 100 - \% Viability$$

Cytotoxic effects of candidate herbal extracts are measured by calculating IC50 (Drug concentration that exerts 50% inhibition) of five novel herbal extracts on ACHN and A2780/cp. This is achieved by using MTT assay as a laboratory test and a standard colorimetric assay (an assay which measures changes in color) for measuring cellular proliferation cytotoxicity.

TABLE 3
SHOWS CYTOTOXIC PROPERTY
OF FIVE CANDIDATE HERBS

| | |
|---|---|
| S. | IC50* |

| No. | Drugs/Herbs | ACHN | HF2 | A2780-cp |
|---|---|---|---|---|
| 1. | Morus alba | 79.325 | 175.774 | 80.663 |
| 2. | Musa sapientum | 67.967 | 178.465 | 77.982 |
| 3. | Arnebia decumbens | 70.932 | 180.776 | 75.254 |
| 4. | Arnebia echioides | 97.665 | 145.932 | 96.912 |
| 5. | Arnebia | 74.973 | 156.445 | 69.721 |
| | linearifoliaDC | | | |
| 6. | Doxorubicin | 3.918 | 1.306 | |
| 7. | Cisplatin | | | 42.489 |

*IC₅₀ of herbal extracts (μg/ml) and IC₅₀ of doxorubicin and cisplatin (μM)

With respect to Table 3, the novel herbal extracts showed cytotoxic effects on A2780/cp and ACHN cell lines with IC50 ranging from 69.7 μg/ml to 97.6 μg/ml.

After subtracting solvent toxicity, the concentration giving 50% inhibition (IC50) is determined for the tested compounds by Probit Analysis (a statistical treatment of the sigmoid response curve). SPSS program version 10.0 is used to calculate IC50. To analyze the interaction between herbal extracts and the anticancer drugs, Zheng-Jun Jin method and CI method are applied. In Zheng-Jun Jin method “Q” value is introduced, according to which the interaction between two agents can be classified as Antagonistic effect (Q≦0.85), Additive effect (0.85≦Q<1.15), and Synergistic effect (Q≧1.15). The amount of Q is calculated by the following equation:

Q=Ea+b/(Ea+Eb−Ea×Eb)

wherein Ea+b is the average effect of combination treatment of a and b and Ea and Eb are the effect of drug a and b separately, respectively.

CI method is based on the combination index theorem and present a quantitative measure based on the mass-action law of the degree of drug interaction in terms of Synergism (CI>1), Additive (CI=1) and Antagonism (CI<1) for a particular endpoint of the effect measurement. CI is calculated from the following equation:

n(CI)x=Σnj=1(D)j/(D50)j

wherein D and D50 are defined as Dose and Median-effect dose, respectively. The dose which produces 50% effect is known as IC50.

The various resulted values of Q, CI and CSI for different herbal extracts with cisplatin using A2780-cp cell line are shown in Table 4.

TABLE 4
SHOWS SYNERGISTIC PROPERTY OF CANDIDATE HERBAL
EXTRACTS WITH CISPLATIN ON A2780-cp CELL LINE

| | IC₅₀ (μM) | | | |
|---|---|---|---|---|
| Cisplatin | 42.489 | Q* | CI** | CSI*** |
| Cisplatin + Morus alba (100 μg/ml) | 10.197 | 1.324 | 0.448 | 4.166 |
| Cisplatin + Morus alba (50 μg/ml) | 12.025 | 1.281 | 0.451 | 3.533 |
| Cisplatin + Morus alba (25 μg/ml) | 15.144 | 1.233 | 0.454 | 2.805 |
| Cisplatin + Morus alba (12.5 μg/ml) | 17.127 | 1.202 | 0.456 | 2.480 |
| Cisplatin + Musa sapientum (100 μg/ml) | 11.601 | 1.278 | 0.450 | 3.662 |
| Cisplatin + Musa sapientum (50 μg/ml) | 14.829 | 1.228 | 0.452 | 2.865 |
| Cisplatin + Musa sapientum (25 μg/ml) | 15.134 | 1.207 | 0.453 | 2.807 |
| Cisplatin + Musa sapientum (12.5 μg/ml) | 16.114 | 1.162 | 0.455 | 2.636 |
| Cisplatin + Arnebia decumbens (100 μg/ml) | 10.603 | 1.328 | 0.449 | 4.007 |
| Cisplatin + Arnebia decumbens (50 μg/ml) | 12.280 | 1.296 | 0.450 | 3.460 |
| Cisplatin + Arnebia decumbens (25 μg/ml) | 14.545 | 1.261 | 0.454 | 2.921 |
| Cisplatin + Arnebia decumbens (12.5 μg/ml) | 15.274 | 1.187 | 0.456 | 2.781 |
| Cisplatin + Arnebia echioides (100 μg/ml) | 11.873 | 1.373 | 0.447 | 3.578 |
| Cisplatin + Arnebia echioides (50 μg/ml) | 12.756 | 1.303 | 0.449 | 3.330 |
| Cisplatin + Arnebia echioides (25 μg/ml) | 16.487 | 1.216 | 0.453 | 2.577 |
| Cisplatin + Arnebia echioides (12.5 μg/ml) | 17.705 | 1.159 | 0.454 | 2.399 |
| Cisplatin + Arnebia linearifolia DC (100 μg/ml) | 9.131 | 1.260 | 0.451 | 4.653 |
| Cisplatin + Arnebia linearifolia DC.(50 μg/ml) | 10.068 | 1.232 | 0.453 | 4.220 |
| Cisplatin + Arnebia linearifolia DC (25 μg/ml) | 15.044 | 1.213 | 0.456 | 2.824 |
| Cisplatin + Arnebia linearifoliaDC (12.5 μg/ml) | 16.679 | 1.185 | 0.458 | 2.547 |

*Antagonistic effect (Q ≦ 0.85), Additive effect (0.85 ≦ Q < 1.15), and Synergistic effect (Q ≧ 1.15)
**Combination Index; Antagonism (CI > 1), Additive (CI = 1) and Synergism (CI < 1)
***Chemosensitizing index = IC₅₀ cisplatin/IC₅₀ cisplatin plus herbal extract

With respect to Table 4 resulted “Q” values are greater than 1.15 showing synergism. The “CI” values are less than 1, again showing synergism. The values of Chemosensitizing index (CSI) for Arnebia decumbens, Morus alba in concentrations of 100 μg/ml are greater than 4. The values of Chemosensitizing index (CSI) for Arnebia linearifolia DC. in concentrations of 100 μg/ml and 50 μg/ml are greater than 4. The values of Chemosensitizing index (CSI) Musa sapientum in concentrations of 100 μg/ml, Arnebia decumbens in concentrations of 50 μg/ml and Arnebia echioides in concentrations 100 and 50 μg/ml are greater than 3. The values of Chemosensitizing index (CSI) for other herbal extract concentrations in combination with cisplatin on A2780/cp cell line are greater than 2.3.

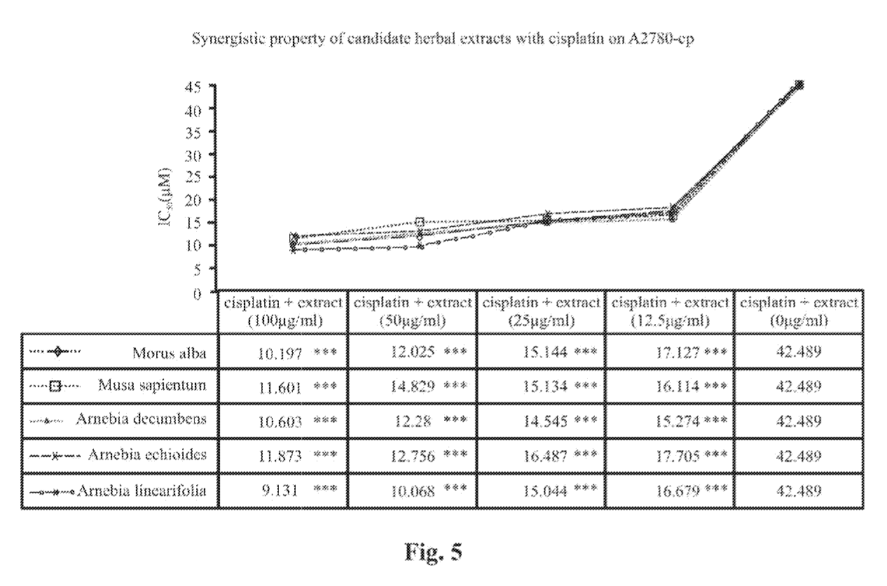

FIG. 5 shows a graphical representation of the synergistic property of candidate herbal extracts with cisplatin using A2780-cp cell line according to one embodiment herein. With respect to FIG. 5, the IC50 values generated by single anticancer drug (cisplatin) treatments are extremely higher than several concentrations of five herbal extracts in combination with cisplatin on A2780/cp. The means of cytotoxic percents induced by single anticancer drug treatments and combinational treatments are calculated and the results are compared by one-way and repeated measures Analysis of Variance (ANOVA) method. It is seen that, the cytotoxic percents induced by cisplatin alone is extremely significant in comparison with cytotoxic percents induced by combinational treatments on A2780/cp. The stars show the rate of signification between cisplatin alone and several concentrations of five herbal extracts in combination with cisplatin. It is notable that p value is calculated by GrophPad Instat—[DATASET1.ISD] version 3.00. The *** is related to p<0.001which is extremely significant.

The various resulted values of Q, CI and CSI for different herbal extracts with doxorubicin using ACHN cell line are shown in Table 5.

TABLE 5
SHOWS SYNERGISTIC PROPERTY OF CANDIDATE HERBAL
EXTRACTS WITH DOXORUBICIN ON ACHN CELL LINE

| | IC₅₀ (μM) | | | |
|---|---|---|---|---|
| Doxorubicin | 3.918 | Q* | CI** | CSI*** |
| Doxorubicin + Morus alba (100 μg/ml) | 0.543 | 1.350 | 0.539 | 7.215 |
| Doxorubicin + Morus alba (50 μg/ml) | 0.813 | 1.292 | 0.551 | 4.819 |
| Doxorubicin + Morus alba (25 μg/ml) | 1.115 | 1.239 | 0.577 | 3.513 |
| Doxorubicin + Morus alba (12.5 μg/ml) | 1.326 | 1.200 | 0.627 | 2.954 |
| Doxorubicin + Musa sapientum (100 μg/ml) | 0.667 | 1.288 | 0.541 | 5.874 |
| Doxorubicin + Musa sapientum (50 μg/ml) | 0.874 | 1.272 | 0.543 | 4.482 |
| Doxorubicin + Musa sapientum (25 μg/ml) | 1.245 | 1.253 | 0.571 | 3.146 |
| Doxorubicin + Musa sapientum (12.5 μg/ml) | 1.337 | 1.222 | 0.599 | 2.930 |
| Doxorubicin + Arnebia decumbens (100 μg/ml) | 0.884 | 1.347 | 0.528 | 4.432 |
| Doxorubicin + Arnebia decumbens (50 μg/ml) | 1.088 | 1.314 | 0.533 | 3.601 |
| Doxorubicin + Arnebia decumbens (25 μg/ml) | 1.478 | 1.282 | 0.552 | 2.650 |
| Doxorubicin + Arnebia decumbens (12.5 μg/ml) | 1.619 | 1.244 | 0.569 | 2.420 |
| Doxorubicin + Arnebia echioides (100 μg/ml) | 0.701 | 1.398 | 0.529 | 5.589 |
| Doxorubicin + Arnebia echioides (50 μg/ml) | 0.878 | 1.373 | 0.536 | 4.462 |
| Doxorubicin + Arnebia echioides (25 μg/ml) | 1.384 | 1.340 | 0.570 | 2.830 |
| Doxorubicin + Arnebia echioides (12.5 μg/ml) | 1.588 | 1.291 | 0.593 | 2.467 |
| Doxorubicin + Arnebia linearifoliaDC (100 μg/ml) | 0.892 | 1.347 | 0.527 | 4.392 |
| Doxorubicin + Arnebia linearifoliaDC (50 μg/ml) | 1.043 | 1.320 | 0.545 | 3.756 |
| Doxorubicin + Arnebia linearifoliaDC. (25 μg/ml) | 1.206 | 1.293 | 0.556 | 3.248 |
| Doxorubicin + Arnebia linearifoliaDC. (12.5 μg/ml) | 1.637 | 1.271 | 0.560 | 2.393 |

*Antagonistic effect (Q ≦ 0.85), Additive effect (0.85 ≦ Q < 1.15), and Synergistic effect (Q ≧ 1.15)
**Combination Index; Antagonism (CI > 1), Additive (CI = 1) and Synergism (CI < 1)
***Chemosensitizing index = IC₅₀ doxorubicin/IC₅₀ doxorubicin plus herbal extract

With respect to Table 5 resulted “Q” values are greater than 1.15 showing synergism. The “CI” values are less than 1, again showing synergism. The values of Chemosensitizing index (CSI) for Morus alba, Musa sapientum and Arnebia echioides in concentrations of 100 and 50 μg/ml and Arnebia decumbens and Arnebia linearifolia DC. in concentrations 100 μg/ml are greater than 4. The values of Chemosensitizing index (CSI) for other herbal extract concentrations in combination with doxorubicin on ACHN cell lines are greater than 2.3.

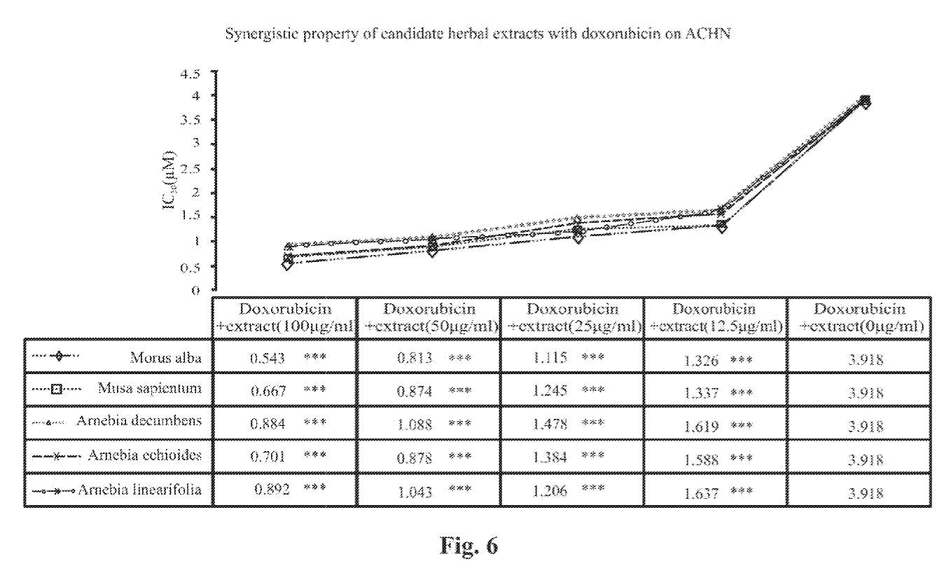

FIG. 6 shows a graphical representation of the synergistic property of candidate herbal extracts with doxorubicin using ACHN cell line according to one embodiment herein. With respect to FIG. 6, the IC50 values generated by single anticancer drug treatments (doxorubicin) are extremely higher than several concentrations of five herbal extracts in combination with doxorubicin on ACHN. The means of cytotoxic percents induced by single anticancer drug treatment and combinational treatments are calculated and the results are compared by one-way and repeated measures Analysis of Variance (ANOVA) method. It is seen that, the cytotoxic percents induced by doxorubicin alone is extremely significant in comparison with cytotoxic percents induced by combinational treatments on ACHN. The stars show the rate of signification between doxorubicin alone and several concentrations of five herbal extracts in combination with doxorubicin. It is notable that p value is calculated by GrophPad Instat—[DATASET1.ISD] version 3.00 and *** is related to P<0.001 which is extremely significant.

The various resulted values of Q, CI and CSI for different herbal extracts with doxorubicin using HF2 cell line are shown in Table 6.

TABLE 6
SHOWS ANTAGONISTIC PROPERTY OF CANDIDATE HERBAL
EXTRACTS WITH DOXORUBICIN ON HF2 CELL LINE

| | IC₅₀ (μM) | | | |
|---|---|---|---|---|
| Doxorubicin | 1.306 | Q* | CI** | CSI*** |
| Doxorubicin + Morus alba (100 μg/ml) | 4.091 | 0.497 | 1.455 | 0.319 |
| Doxorubicin + Morus alba (50 μg/ml) | 5.015 | 0.346 | 1.451 | 0.260 |
| Doxorubicin + Morus alba (25 μg/ml) | 5.344 | 0.297 | 1.450 | 0.244 |
| Doxorubicin + Morus alba (12.5 μg/ml) | 5.625 | 0.275 | 1.449 | 0.232 |
| Doxorubicin + Musa sapientum (100 μg/ml) | 5.182 | 0.558 | 1.451 | 0.252 |
| Doxorubicin + Musa sapientum (50 μg/ml) | 7.024 | 0.466 | 1.447 | 0.185 |
| Doxorubicin + Musa sapientum (25 μg/ml) | 7.462 | 0.389 | 1.446 | 0.175 |
| Doxorubicin + Musa sapientum (12.5 μg/ml) | 7.795 | 0.296 | 1.444 | 0.167 |
| Doxorubicin + Arnebia decumbens (100 μg/ml) | 4.622 | 0.453 | 1.453 | 0.282 |
| Doxorubicin + Arnebia decumbens (50 μg/ml) | 6.346 | 0.382 | 1.448 | 0.205 |
| Doxorubicin + Arnebia decumbens (25 μg/ml) | 6.843 | 0.306 | 1.447 | 0.190 |
| Doxorubicin + Arnebia decumbens (12.5 μg/ml) | 7.201 | 0.257 | 1.446 | 0.181 |
| Doxorubicin + Arnebia echioides(100 μg/ml) | 5.338 | 0.492 | 1.450 | 0.244 |
| Doxorubicin + Arnebia echioides (50 μg/ml) | 6.037 | 0.385 | 1.449 | 0.216 |
| Doxorubicin + Arnebia echioides (25 μg/ml) | 6.467 | 0.306 | 1.448 | 0.201 |
| Doxorubicin + Arnebia echioides(12.5 μg/ml) | 7.073 | 0.268 | 1.447 | 0.184 |
| Doxorubicin + Arnebia linearifoliaDC (100 μg/ml) | 5.359 | 0.444 | 1.450 | 0.243 |
| Doxorubicin + Arnebia linearifoliaDC. (50 μg/ml) | 7.189 | 0.316 | 1.446 | 0.181 |
| Doxorubicin + Arnebia linearifoliaDC. (25 μg/ml) | 7.428 | 0.285 | 1.446 | 0.175 |
| Doxorubicin + Arnebia linearifoliaDC. (12.5 μg/ml) | 7.931 | 0.257 | 1.445 | 0.164 |

*Antagonistic effect (Q ≦ 0.85), Additive effect (0.85 ≦ Q < 1.15), and Synergistic effect (Q ≦ 1.15)
**Combination Index; Antagonism (CI > 1), Additive (CI = 1) and Synergism (CI < 1)
***Chemosensitizing index = IC₅₀ doxorubicin/IC₅₀ doxorubicin plus herbal extract

With respect to Table 6 resulted “Q” values are less than 0.85 showing antagonism. The “CI” values are more than 1, again showing antagonism. The values of Chemosensitizing index (CSI) for all herbal extract concentrations in combination with doxorubicin on HF2 cell lines are lower than 0.31.

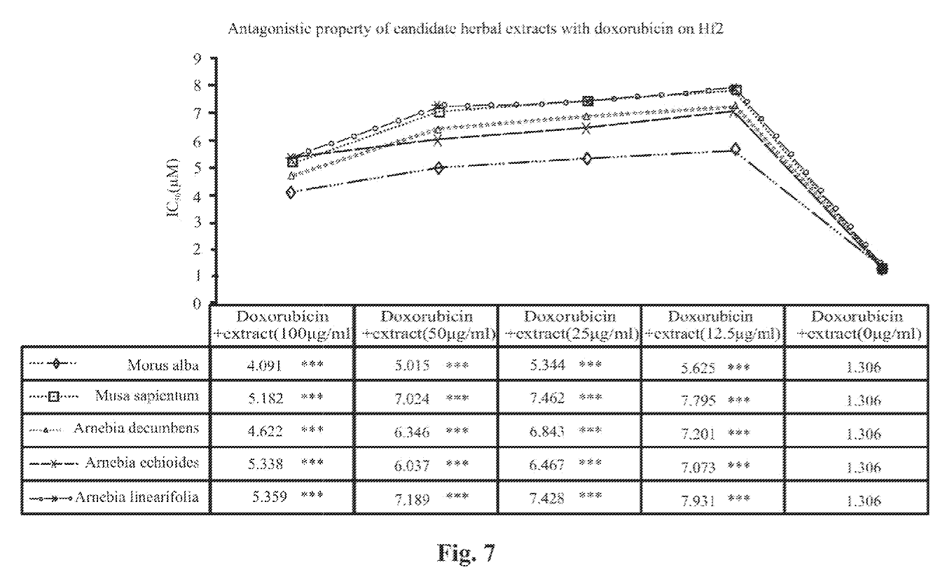

FIG. 7 shows a graphical representation of the antagonistic property of candidate herbal extracts with doxorubicin using HF2 cell line according to one embodiment herein. With respect to FIG. 7, the IC50 values generated by single anticancer drug treatment (doxorubicin) are extremely lower than several concentrations of five herbal extracts in combination with doxorubicin on HF2. The means of cytotoxic percents induced by single anticancer drug treatment and combinational treatments are calculated and the results are compared by one-way and repeated measures Analysis of Variance (ANOVA) method. The cytotoxic percents induced by doxorubicin alone are extremely significant in comparison with cytotoxic percents induced by combinational treatments on HF2. The stars show the rate of signification between doxorubicin alone and several concentrations of five herbal extracts in combination with doxorubicin. It is notable that P value is calculated by GraphPad Instat—[DATASET1.ISD] version 3.00. The *** is related to P<0.001 which is extremely significant.

The results show that all five herbal extracts synergistically increase cytotoxic effects of cisplatin and doxorubicin on A2780/cp and ACHN in dose dependent manner. Since MDR efflux pumps are expressed on the cell membranes of HF2 cells and this cell line is not resistant to doxorubicin, all five herbal extracts antagonistically decrease cytotoxic effect of doxorubicin on HF2 as normal cell. Both resulted “Q” and “CI” values calculated by two mentioned methods show synergistic effect of combination treatment in A2780/cp and ACHN, and effect of combination treatment on HF2 follows an antagonistic profile.

Hemolytic test is done to determine biocompatibility of herbal extracts. Hemolytic test is done for five herbal extracts to measure their property of inducing hemolysis. The hemolytic test is performed in 96-well plates. Wherein, 50 μl of 0.85% NaCl solution containing 10 mM CaCl₂ is put in each well. The first column acts as negative control that contains only 50 μl of saline solution. Then, 50 μl of 0.2% Triton X-100 (in 0.85% saline) is put in the first well of second column of plate to obtain 100% hemolysis. The first well in each respective group acts as control containing the solvent and the vehicle (2% DMSO). Then, 50 μl of herbal extracts diluted in half are added to the test sets. The herbal extracts are tested at concentrations ranging from 3.25 to 200 μg/ml. Then, 50 μl of a 2% suspension of sheep erythrocytes in 0.85% saline containing 10 mM CaCl₂ is added to each well. Each single dose and combination is in triplicate in each assay. After incubation at room temperature for 30 min, and centrifugation for 3 min (750 g), the supernatant is removed and the liberated hemoglobin is measured spectroscopically at absorbance of 540 nm.

Finally, it is concluded from hemolytic test that candidate herbal extracts did not induce hemolysis similar to negative control, which is further evidence for lack of general toxicity of the selected herbal extracts.

Selected herbal extracts in combination with doxorubicin and cisplatin have been used to sensitize ACHN (human renal adenocarcinoma) and A2780/cp (human ovarian carcinoma, resistant to cisplatin) and overcome MDR phenomenon. The obtained results introduce a selectivity effect of mentioned herbal extracts in inducing cytotoxicity on cancerous cell lines and reversing of undesirable MDR phenomenon.

The various embodiments of the present invention are designed and carried out on the basis of cheminformatics method. In vitro results confirmed the predicted findings. By using a novel approach in combining cheminformatics, intensive literature handling, together with correlation of biologic data to search for the desired biologic activity in the domain of natural products that are not explored before.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A method of selecting candidate herbs for combination drug therapy, the method comprising the steps of:
enlisting a pluralities of herbs with a property to treat a desired disease from a plant database;
classifying the collected pluralities of herbs into synergistic plants; selecting bioactive compounds from the synergistic plants;
categorizing the selected bioactive compounds into synergistic compounds;
acquiring Simplified Molecular Input Line Entry Specification (SMILES) based on chemical structures of the synergistic compounds;
conducting a similarity search using the acquired SMILES as a data query to find and classify compounds that are similar to categorized synergistic compounds into similar synergistic compounds;
classifying the similar synergistic compounds that are derived from plant source into similar plant synergistic compounds;
checking the novelty of the herbs containing similar herbal synergistic compounds in treating a desired disease;
confirming a synergistic property of the herbs containing similar herbal synergistic compounds through a bioassay process to determine the synergistic property of the herbs containing similar herbal synergistic compounds in treating the desired disease;
grouping the herbs containing similar herbal synergistic compounds into candidate herbs based on the determined synergistic property with respect to the desired disease.

2. The method according to claim 1, wherein the similar synergistic compounds are searched and acquired using an algorithm.

3. The method according to claim 2, wherein the algorithm is derived based on a cheminformatics tool.

4. The method according to claim 1, wherein the plant database includes previous findings.

5. The method according to claim 1, wherein the desired disease is cancer.

6. The method according to claim 1, wherein the step of collecting the pluralities of herbs with a property to treat a desired disease involves searching the pluralities of herbs with the property to treat the desired disease from the previous findings.

7. The method according to claim 6, wherein the previous findings include available books and articles.

8. The method according to claim 1, wherein the step of checking the novelty of the herbs containing similar herbal synergistic compounds in treating the desired disease includes performing a literature survey.

9. The method according to claim 1, wherein the step of confirming the synergistic property of the herbs containing similar herbal synergistic compounds involves performing in vitro assay to confirm a promising profile of the herbs containing similar herbal synergistic compounds in treating the desired disease.

10. A method of selecting novel anti-cancer herbs using cheminformatic tools, the method comprising the steps of:
Enlisting a plurality of herbs having synergistic activity with anti-cancer compounds by searching in plant databases and previous findings;
categorizing the collected plurality of herbs into synergistic plants(SP);
selecting bioactive compounds from the synergistic plants (SP) and categorizing as synergistic compounds collection(SCC);
performing a similarity search using the selected bioactive compounds categorized as SCC in available databases to obtain similar synergistic compounds (SSC);
searching herbal source for the similar synergistic compounds (SSC);
categorizing the compounds having the similar herbal source as similar herbal synergistic compounds (SHSC);
checking for novelty of SHSC in a field of cancer treatment;
categorizing the novel SHSC in the field of cancer treatment novel candidate herbs (NCH); and
performing in vitro bioassay to confirm the synergistic activity of the NCH as anti-cancer agents.

11. The method according to claim 10, wherein the novel candidate herbs (NCH) are Morus alba, Musa sapientum, Arnebia decumbens, Arnebia echioides, Arnebia linearifolia DC.

12. The method according to claim 10, wherein Doxorubicin and cisplatin used as positive control drugs

13. The method according to claim 10, wherein the bioactive compounds selected from the synergistic plants (SP) and categorized into a synergistic compounds collection (SCC) are kaempferol, epicatechin and juglone.

14. The method according to claim 10, wherein the compounds selected and categorized as similar synergistic compounds (SSC) are morin, leucocyanidin, arnebin-7 and arnebin.

15. A composition for treating cancer comprising:
herbal extracts of Morus alba, Musa sapientum, Arnebia decumbens, Arnebia echioides, Arnebia linearifolia DC in combination with anti-cancer agents.

16. The composition according to claim 15, wherein the anti-cancer agents are cisplatin and doxorubicin.
